(19) United States
(12) Patent Application Publication
(10) Pub. No.: US 2008/0175871 A1
(43) Pub. Date: Jul. 24, 2008

(54) ELICITING IMPROVED IMMUNE RESPONSES

(75) Inventors: Arthur Randall BRADWELL, Birmingham (GB); Graham Peter MEAD, Birmingham (GB)

Correspondence Address: WILMERHALE/NEW YORK, 399 PARK AVENUE, NEW YORK, NY, 10022, US

(73) Assignee: THE BINDING SITE LIMITED

(21) Appl. No.: 12/017,611

(22) Filed: Jan. 22, 2008

Related U.S. Application Data
(62) Division of application No. 09/547,199, filed on Apr. 12, 2000.

(30) Foreign Application Priority Data
Apr. 13, 1999 (GB) 9908263.8

(51) Int. Cl. A61K 39/00; A61P 37/00

(52) U.S. Cl. 424/278.1

(57) ABSTRACT

An improved immune response is achieved in an animal by administering, for example injecting, a cocktail of peptides that are similar to, but different from, a target peptide against which it is desired to create antibodies. The similar peptides preferably have the same peptide sequence as the target peptide, but with one or more amino acids substituted for a chance-selected, random, amino acid from a group of allowable amino acids. The peptides may be present on a multi-branch peptide.

This application is a divisional of U.S. Ser. No. 09/547,199, filed on Apr. 12, 2000, which claims priority to the application GB 9908263.8, filed on Apr. 13, 1999.

### BACKGROUND

Tolerance to autoantigens occurs during B and T lymphocyte development in the bone marrow and thymus respectively. Self-reacting differentiating B or T cells are normally rendered anergic or deleted from the repertoire to prevent future onset of autoimmune disease.

One previously described strategy for overcoming tolerance and eliciting an immune response against an autologous protein comprises co-injecting the autologous protein and the “same” protein isolated from a different species (“a foreign protein”) into the patient. An example of this is injecting a combination of the human and bovine forms of a protein into the patient along with an adjuvant. The bovine form of the protein is, of course, not absolutely exactly the same as the human form.

During the resulting immune response, the patient will produce antibodies to both the bovine and the human forms of the injected protein. The reasons for the success of this mechanism are not fully understood. One hypothesis is that the B lymphocytes in the patient's circulation are reactive to epitopes on both the native and foreign proteins (cross-reactivity). The B-cells bind the cross-reacting epitopes and present autologous protein to CD4+ T-cells and stimulate an autoreactive T-cell response. One possibility is that previously-anergised self-reacting T-helper cells can be stimulated by the presentation of cross-reacting antigens by the B-cells.

The method relies on the availability of whole (or at least significant fragments of) biologically active domains of human and non-human sources of the target protein in order to elicit an effective immune response against the target protein, and obtaining these can be expensive and time consuming. The strategy may not always be effective, particularly if the differences between the two proteins are so large that they limit the cross-reaction of antibodies raised against the one (or alternatively) they may be too similar to get a good response of generated antibodies attacking the target protein). The method is generally used with an adjuvant, frequently Freund's Incomplete Adjuvant (FIA), or Freund's Complete Adjuvant (FCA) in small doses.

An alternative to immunotherapy is the use of drugs which lower the level of the protein that is causing the adverse symptoms. Generally, however, it is found that the effect of such drugs diminishes with repeated courses.

If a tumour is the cause of the elevated protein level, excision of the tumour is not always possible, especially after the tumour has metastasised.

In certain cases, if the cancer is the trigger for the elevated level of protein, radiotherapy and cytotoxic agents can also be ineffective.

There is a need, therefore, for a composition which is capable of eliciting an immune response to a molecule quickly, cheaply and effectively.

### SUMMARY OF THE INVENTION

The invention relates to pharmaceutical preparations (and uses thereof) for use in eliciting an immune response in a patient, the immune response being directed to one or more target molecules, especially one or more target proteins, and to treatments using them. The invention is applicable to any pharmaceutical preparations containing proteins or peptides, for use in eliciting immune responses. The invention is especially but not exclusively applicable to pharmaceutical preparations for use in eliciting immune responses directed to autologous proteins which are present in the patient. The term “protein” will be used herein to include proteins and other molecules which contain sequences of amino acids, such as, but not limited to, peptides, lipoproteins or glycoproteins.

Those skilled in the art will appreciate that immunotherapy can be used to treat various diseases and their associated symptoms. The treatment can either involve exposing the patient to pre-prepared effector molecules or cells, or stimulating the patient's immune system to produce effector molecules and/or cells directed at the appropriate target(s). This type of treatment is especially applicable if the immune response is directed to foreign proteins or toxins but it may also be used if the target proteins are native to the patient (autologous).

Autologous proteins may usefully be the target of immunotherapy if they are the cause of a disease (e.g. elevated hormone levels) or associated with the disease (e.g. proteins expressed on cancerous cells). Many such diseases are currently untreatable but we believe may be improved by immunotherapy if the immune system could be made to respond appropriately by overcoming the patient's tolerance to the self-antigens (autoantigens).

### BRIEF DESCRIPTION OF THE FIGURES

There now follows by way of example only, a detailed description of the invention with reference to the accompanying drawings of which:

FIGS. 1A and 1B show a multi antigenic peptide systems (MAP) which may be used in the composition according to the present invention.

FIG. 2 schematically shows a target molecule and a medicament comprising four modified target amino acid sequences.

FIGS. 3A and 3B show kits incorporating the invention.

FIG. 4 illustrates four variants on a target molecule, which variants are active ingredients in a composition to be administered.

### DETAILED DESCRIPTION

According to a first aspect of the invention, there is provided a composition for use in eliciting an immune response to a target molecule, the composition comprising an immunological adjuvant and one or more proteins (as hereinbefore defined) in which each of any one or more of the amino acids constituting one or more of the proteins is replaced with another amino acid selected from a mixture of at least two amino acids. Suitably, the target molecule comprises a protein/peptide, a lipoprotein, a glycoprotein or a carbohydrate. Preferably the proteins have an amino acid sequence substantially equivalent to at least part of the primary sequence of the target molecule but include a residue change at one or more positions in the protein. The changed residue is substituted with an amino acid from a pool of two or more amino acids such that the pharmaceutical preparation comprises a plurality of different proteins. The term “amino acids” is intended to include amino acids and their analogues.

Preferably, each protein has at least one residue substituted. Suitably, the residue in the protein chain is substituted for one of the naturally occurring amino acids, or any other chemical moiety that could be substituted for an amino acid in this context. Thus a residue from the list: glycine (G), alanine (A), valine (V), leucine (L), isoleucine (I), serine (S), cysteine (C), threonine (T), methionine (M), proline (P), histidine (H), arginine (R), lysine (K), glutamic acid (E), aspartic acid (D), glutamine (Q), asparagine (N), tyrosine (Y), tryptophan (W), phenylalanine (F), may be substituted for a different residue from the same list.

Preferably, the identity of the substituting amino acid is chosen randomly, for example by using a mixture of amino acids at the appropriate stage in the synthesis of a peptide such that the amino acids in the mixture are substituted into the proteins at equal frequency. Ideally, all the proteins (as hereinbefore defined) in the mixture include random residue substitutions. Preferably, the proteins (as hereinbefore defined) are synthesized and presented on a multiple antigen peptide system (MAP) such as a lysine web.

Each peptide may be, for example, 10 to 50 amino acids long, such as 20 to 30 amino acids long, and suitably 25 to 30 amino acids long. Each peptide may be approximately 30 amino acids long ±5,4,3, or 2 amino acids, most preferably ±3 amino acids. Preferably, the adjuvant is Freund's Complete Adjuvant (FCA). Alternatively the adjuvant may be Freund's Incomplete Adjuvant (FIA), or any other suitable adjuvant. The target molecule may be a protein autologous to the patient's system. For example, the target molecule may be present in the patient's serum. Alternatively, or additionally, the target molecule may be membrane-bound on the surface of a cell, for example a cancerous or virally-infected cell. The target could be a protein associated with a cancerous cell, or some other unwanted protein. The target molecule could additionally be present on a foreign parasite or microbe. The invention could have applications in prophylactic treatment as well as in treating existing infections and diseases. The present invention in one embodiment allows a cheaper and more universally effective treatment of elevated levels of autologous proteins in the patient. The proteins (as hereinbefore defined) may be synthesized quickly and the random substitution can be performed by known techniques. Subsequent to the first administration of a composition according to the present invention, booster injections of the composition can be given to the patient.

According to a second aspect of the invention, there is provided a method of manufacture of a medicament including the pharmaceutical composition according to the first aspect of the invention, the method comprising a first step of producing a mixture of peptides in which each of any one or more of the amino acids constituting at least one of the peptides is replaced with another amino acid selected from a mixture of at least two amino acids during the production of the or each peptide, and a second step of adding an immunological adjuvant.

According to another aspect of the invention we provide a method of eliciting an immunological response to a target molecule comprising injecting or otherwise administering a preparation having a protein which is substantially the same as at least a part of the target molecule except that at least one amino acid of the administered protein, referred to as modulated protein, is different at a modulation site in its amino acid sequence to the amino acid that is present in the target molecule at the position of the modulation site in the amino acid sequence of the target molecule.

Preferably the modulation protein is administered in vivo to an animal, for example a mammal, such as a human. The target molecule (or said at least a part of it) may also be administered along with the modulated protein, possibly in the same liquid/medicament/vaccine.

Preferably the method comprises administering a plurality of modulated proteins including at least as first modulated protein having a first varied amino acid at a first modulation site in its peptide sequence, and a second varied protein having a second varied amino acid at a second modulation site in its peptide sequence.

The first and second varied amino acids may be different from each other, or they may be the same (but with the position of the modulation site different). The first and second modulation sites may be at different positions in the peptide sequence of the target molecule (or at the same position in the peptide sequence), and the first and second varied amino acids may be the same as each other or different from each other.

There may of course be a third, or further, modulation site(s) in the peptides.

Preferably the varied proteins (targetted against a particular target molecule) have the same peptide sequence as said at least part of the target molecule except for one different amino acid at one place in the amino acid sequence of the varied protein. Alternatively there could be more than one difference in amino acid sequence (e.g. 2 or 3 base differences), with the rest of its sequences being the same. Each of the modulated proteins targetted against the same target molecule may have the same amino acid sequence as each other, but with one or two differences in amino acids (or three differences).

Preferably the method comprises having the preparation have a plurality of differently modulated proteins, each having the same modulation site in the peptide sequence, but with modulated amino acids at the modulation site that are different from each other. There may be 2-20 different variations on the modulated amino acid, preferably 2-10, and most preferably 4-8. There may be at least 2, 3, 4, 5, 6, 7, 8, 9, 10, 11, 12, 13, 14, 15, 16 or more, different modulated proteins in the preparation.

Preferably the method comprises administering at least 3, 4, 5, 6, 7, 8, 9, 10, 11, 12 or more modulated proteins. Preferably 20 or less modulated proteins are administered. There may be 20, 15, 10, or 8, or less different amino acids in the modulated proteins that are administered. There may be 20, 15, 10, 8 or less amino acids as the variation/varied amino acid and/or 20, 15, 10, 8, or less types of amino acid in the modulated proteins of the preparation as a whole.

Preferably the preparation is administered intradermally, subcutaneously, intravenously, or intraperitoneally. It may be administered in a primary administration at a first time and in a second booster administration some time later, for example weeks later, possibly about 3, 4, 5, 6 or so weeks later. Further booster administrations of the preparation may be given to the body/person at similar intervals.

Preferably the different varied proteins are present in the same preparation and administered together. Alternatively, we may administer the varied proteins in different administration operations which may be substantially simultaneously, possibly at the same injection/administration site, possibly at different sites on the body, or at time spaced intervals.

The method may comprise identifying two different peptide sequences in the target molecule and administering modified proteins against each of the two different peptide sequences.

The method may comprise identifying a plurality of proteins associated with a disorder or disease and administering a plurality of different varied proteins adapted to cause the production of antibodies targeted against the plurality of proteins.

According to another aspect of the invention we provide a method of producing a composition adapted to elicit an immune response from an immunological system to cause the immunological system to produce antibodies against a target molecule, the method comprising synthesizing a plurality of modulated peptide sequences that are substantially the same as that of at least a part of the target molecule except that they have at least one base, and preferably only one or two bases, different from the target peptide (and from each other), the method comprising taking a first part of the peptide sequence of the modified peptides that is such that the modulated proteins have the same peptide sequence in their first part as each other and/or as the target molecule, and adding or subtracting a mixture of variation amino acids such that the identity of the variation amino acid that joins to any selected first part from the mixture is a matter of chance, thereby producing a mixture of varied peptide sequences which have a common first part and a chance-selected variation amino acid.

A second part of the peptide sequence of the target molecule, identical to the sequence of part of the target molecule and/or to other second parts, may be joined to the chance-selected variation amino acids.

The first and/or second parts may be grown amino acid-by-amino acid under controlled conditions so that the next adding amino acid is well selected and defined (with the varied amino acid being created by a mixture of amino acids in the growth of the peptide chain at the position of the variation), or a pre-prepared length of amino acid sequence may be used to make the modulated proteins. Possibly a pre-prepared first part could be joined to a pre-prepared second part via a randomly/chance selected variation amino acid.

According to another aspect of the invention we provide a composition for use in treatment of humans or mammals, the composition having a plurality of active ingredients including a first active ingredient having a first amino acid sequence and a second active ingredient having a second amino acid sequence, the second amino acid sequence being substantially the same as the first amino acid sequence except that at least one of the amino acids in the sequence is different from the amino acid at the same position in the sequence of the first amino acid sequence; and in which both the first and second amino acid sequences are similar to, but different from, a target amino acid sequence.

Preferably the first and second amino acid sequences differ from the target sequence at the same place in their sequence, but with a different amino acid to each other at that place.

The first and second amino acids may have a second site in their sequence where they differ from each other and/or the target sequence.

Preferably, there are at least 4, and possibly 5, 6, 7, 8, 9, 10, or more, different active ingredients. They may differ from each other at the same position in their amino acid sequence by having different amino acids at said same position.

Each active ingredient may have a length of 10-40 or 50 bases, preferably at least some of them (or all of them) have a length of 20-30 bases. There may only be 1 or 2 differences in amino acids between any two of the active ingredients. The differences between active ingredients may be at the same sites.

The composition may have a first set of active ingredients directed at a first target molecule, and a second set, or subsequent sets, of active ingredients directed at a second, or subsequent, target molecules. The variations between an active ingredient in one set and that in another set are not usually important (because they are eliciting different antibodies to be produced) but the variations in amino acids between active ingredients in the same group should be within the definitions specified above for the first group (although not all of the different groups of active ingredient have to have the same intra-group variation parameters). For example a first group of active ingredients directed against a first protein could have four variations of site x in their peptide chain, whereas active ingredients for a second group could have eight variations at site y.

Preferably the composition has the active ingredients in substantially pure form. There may be substantially no extraneous proteins in the composition apart from the active ingredients (except for possibly an adjuvant).

Preferably the composition includes an adjuvant.

According to another aspect the invention comprises the use of the preparation of an earlier aspect of the invention in a method of treatment.

According to another aspect the invention comprises the use of a preparation according to an earlier aspect of the invention in the preparation of a medicament for the treatment of a disease or disorder with which the target molecule is associated (or target molecules are associated).

According to another aspect the invention comprises a vaccine or medicament comprising a composition according to an earlier aspect of the invention.

According to another aspect the invention comprises the use of the composition according to an earlier aspect of the invention to treat a disease or disorder associated with the target molecule.

According to another aspect the invention comprises a method of treatment of a disease or disorder associated with a known molecule, the method comprising introducing into the patient a composition or vaccine in accordance with an earlier aspect of the invention.

According to another aspect the invention comprises using the method of producing a composition in accordance with an earlier aspect of the invention to produce a medicament or vaccine for use in the treatment of a disease or disorder associated with the target molecule or molecules.

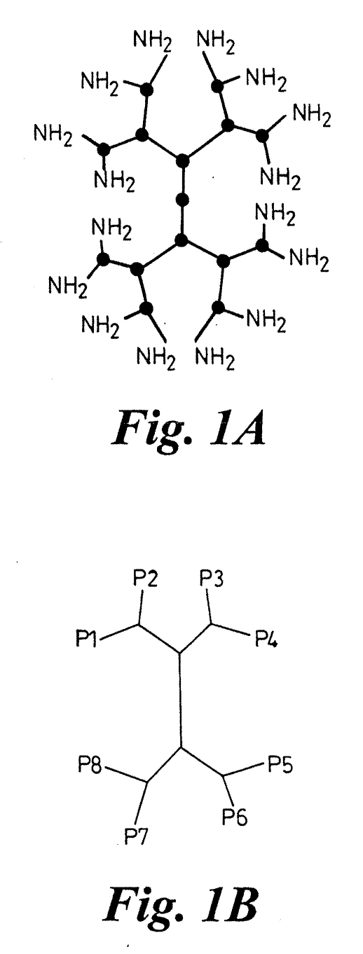

The peptides shown in FIG. 1A are chemically synthesised by the long-established Merrified solid phase method with stepwise addition of amino acids towards the N-terminus. They are synthesised as MAPs so the peptides are grown on a branched lysine core, such as that shown in the drawing. A hexadecavalent web is shown, but divalent, tetravalent and octavalent webs may be used instead of, or in conjunction with, the web shown in the drawing.

Each peptide is 30 amino acids in length and has positions 8 and 23 substituted by random amino acids by using a mix of all twenty of the most common naturally occurring amino acids at these points in the synthesis. A “native” version of the peptide (as a MAP) is also made, without the substitutions, for co-injection.

The soup or cocktail of peptides are mixed 40/60 by volume with Freund's complete adjuvant to give a total volume of approximately 500 μL. The initial immunisation contains 500 μg of each peptide (substituted and non-substituted) and is delivered intradermally in approximately 30 μL volumes at multiple sites. Subsequent booster immunisations are administered similarly (after four weeks) but contain only 200 μg of peptide.

FIG. 1B shows a MAP having peptide chains P1 to P8.

The chains P1 to P8 have a modulation site (the same modulation site) at which the amino acid has been added in chance-selected fashion from a pool or group of different, modulation amino acids. Typically we may use six to twelve different amino acids in the group (e.g. about eight), each of which is different from the amino acid at that position in the target molecule's amino acid sequence. We may have two modulation sites on the branches/chains of peptides attached to the core. It will be appreciated that since the modulation amino acids of the branches P1 to P8 randomly attach from an available group of amino acids there will be a large number of different MAPs since each chain may be different. If there are n chains and r available amino acids there may be n×r different MAPs produced. They will be produced in roughly equal concentrations, depending upon the ease with which each amino acid attaches at the modulation site in question.

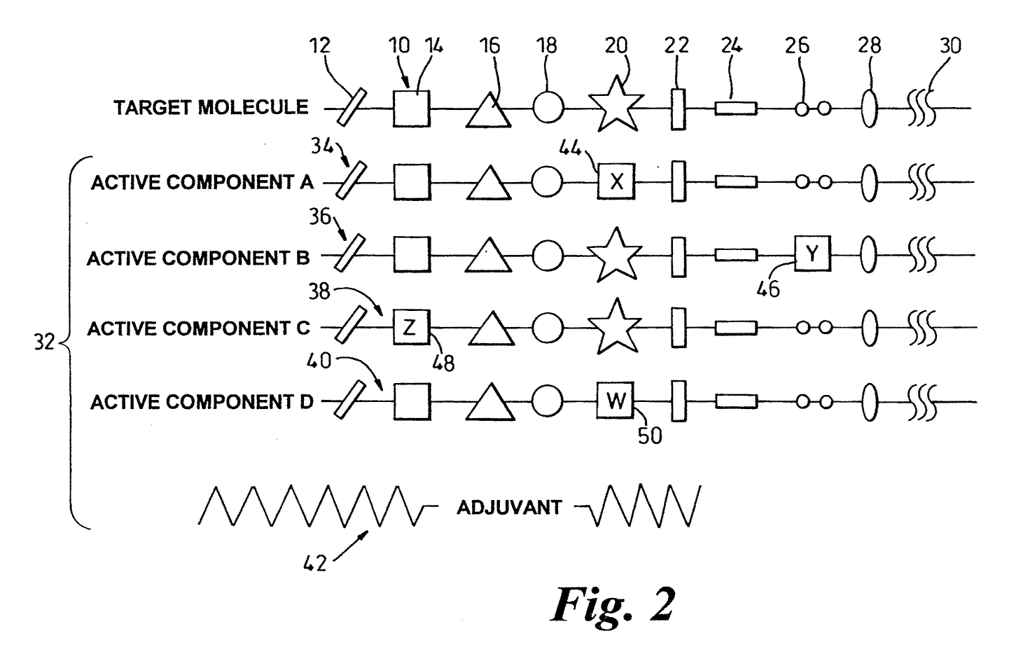

FIG. 2 shows schematically the principle of the invention. A target molecule, referenced 20, is an undesirable protein found in a person, for example an over-expressed hormone. It is desired to have the person have less of target molecule 20 active in their body. The target molecule has different amino acids 12, 14, 16, 18, 20, 22, 24, 26, 28 and 30. A medicament referenced 32, comprises a liquid with active components/ingredients A B C and D and an adjuvant, referenced 34 to 42.

Active component 34 has an amino acid, referenced 44, that is at the place of amino acid 20 in the target molecule that is not the same amino acid 20, but is instead a different amino acid 44.

This “foreign” protein 34 will generate antibodies in the person, which will also attack the target molecule 12.

Similarly in active component 36 the amino acid (referenced 46) at the location of amino acid 26 in the target molecule is not actually the same as the target molecule amino acid 26, but is instead different (the letter Y is not to be taken as indicating, necessary, Tyrosine).

Similarly, the amino acid 48 is not the same as amino acid 14.

The amino acid 50 is different from the amino acid 20 and from the amino acid 44 (and is not necessarily Tryptophan).

In the example of FIG. 2, there are four different active components 34 to 40, but there could be two or more, for example just compounds 34 and 40. It is not essential to have differences at more than one different amino acid site of the plurality of active components different from the target molecule's sequence.

Amino acids 44 to 50 need not all be different from each other, and indeed could be the same amino acid.

We will usually administer the composition/vaccine with an adjuvant, which may be mixed with the active ingredient peptides (or MAP) prior to injection (e.g. just prior, or hours prior, or a day or so prior, or earlier). We may however, provide a mixture of peptides (e.g. MAPs) without an adjuvant being mixed in with them, for example so that a user can add the adjuvant. The peptides will usually be administered in an aqueous solution, for example a buffered saline solution.

The active ingredient peptides (or peptide branches of a MAP) preferably differ from each other (and the target molecule) by 1 base, which for a 20-30 base branch or peptide may be about 5%, or about 4%, or about 3% in terms of sequence identity. We may permit up to 10% variation in sequence identity.

We have found that if we have any more that sixteen branches of peptide on a MAP they tangle themselves up and the MAP does not get quite the optimum or desired immunological response. We therefore prefer to use 4, 8, or 16 branch MAPs (when using a lysine core it is easiest to have the number of branches be a multiple of 4).

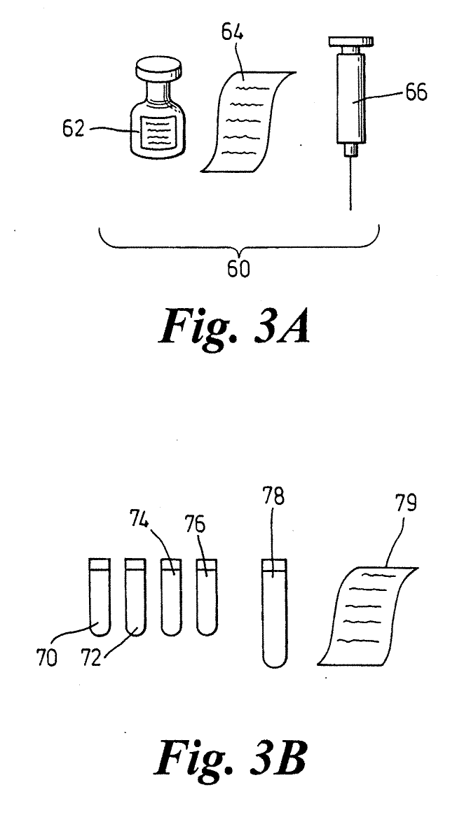

FIG. 3A shows a kit 60 having a vial 62 of medicament (active ingredients plus adjuvant), instructions on use 64, and an optional injection device 66. The kit may have, as shown in FIG. 3B, different active components 70, 72, 74, 76, kept apart from each other an adjuvant 78 and instructions 79. The components 70-76 and the adjuvant may be mixed prior to injection, or they may be injected separately either at substantially the same injection site or at different injection sites (at least two sites).

An example of a medicament and the target protein it is designed to cause antibodies to attack is for the treatment of excess levels of parathyroid hormone. A three-component “soup” of peptides comprise three classes of active ingredient (in addition, usually, to an adjuvant), and these three classes of components are peptides having the amino acid sequences:

| | |
|---|---|
| (i) | |
| SXSEIQLMHNLGKHLNSMERVEWLRKKLQDVHNF | (SEQ ID NO: 1) |
| (ii) | |
| NFVXLGAPLAPRDAGSQRPR | (SEQ ID NO: 2) |
| (iii) | |
| PRKXEDNVLVESHEKSLGEADKADVNVLTKAKSQ | (SEQ ID NO: 3) |

where X is an amino acid that is not the same amino acid found in the parathyroidal hormone at that place in the three sequences.

We could use any two of (i) to (iii), or just one of (i) to (iii).

Preferably the X in soup of peptides that is the medicament is a variety of different amino acids (in fact it does not matter if one of the permutations of X is in fact the same as the corresponding amino acids in the target molecule). For example, X could be any one of two or more, say four to twenty amino acids, so that in fact the medicament has (if say eight different amino acids were used for X) eight different version of active ingredient (i), and eight different versions of active ingredient (ii), and eight different versions of ingredient (iii). The medicament would then have twenty-four different peptide sequences in it as active ingredients, targeted against three different protein sequences.

Of course, we could use a different number of amino acids to substitute for X in (i) as we do in (ii) and/or (iii), so we could have, for example, eight versions of (i), six versions of (ii) and four versions of (iii), present in the soup of peptides of the medicament. All of the versions (or all except one) would be different from the target molecule, and would differ from each other (within a class of active ingredient) by only one peptide.

In one example the amino acids for X are L, F, D, and Q. In another they are L, F, D, Q, P, E, A, and Y.

For melanoma we envisage attacking simultaneously more than one protein associated with it. For example we could provide a medicament having peptides designed to elicit the production of antibodies against from two to all five of the following proteins: MAGE3, MART1, Tyrosinase, Gp100, and TRP2.

Suitable trigger peptides to trigger antibody production that will attack the target molecules are as follows:

For melanoma:

MAGE 3

(SEQ ID NO: 4)

SLQXVFGIELMEVDPIGHLYIFAT

(SEQ ID NO: 5)

YRQXPGSDPACYEFLWGPRALVETS
MART 1

(SEQ ID NO: 6)

MPREDAHFIYGYPKKGHGHSYTTAEEXAGIGILTVILGVL

(SEQ ID NO: 7)

QEKNXEPVVPNAPPAYEKLSAEQSPPPYSP
Tyrosinase

(SEQ ID NO: 8)

MLLAVLYCLXWSFQTSAGHFPRACVSS

(SEQ ID NO: 9)

KQLXEEKQPLLMEKEDYHSLYQSHL
Gp100

(SEQ ID NO: 10)

DGGPCPSGSWSQXRSFVYVWKTWGQYWQVL

(SEQ ID NO: 11)

TIMDQVPFSVXVSQLRALDGGNKHFLRNQP
TRP2

(SEQ ID NO: 12)

NFATGXNECDVCTDQLFGAARPDDPTLI

(SEQ ID NO: 13)

HYYSXRDTLLGPGRPYRAIDFSHQG

where X in the ten peptides classes listed above represents the random substitution, and where the peptides are produced as 8-branch multi-antigenic peptides (i.e. eight branches of a common molecule, e.g. attached to a lysine core).

Vaccine targets and their associated diseases include: PTH in parathyroid carcinoma; Insulin in insulinoma; Hepatitis B virus in chronic viral hepatitis; HIV in AIDS; Tumour necrosis factor ax in rheumatoid arthritis and Crohn's disease; MART 1, Gp100, MAGE 3, tyrosinase & TRP2 in malignant melanoma; C-erbB2 in breast carcinoma and osteosarcoma.

Generally any tumor marker as and when they are identified.

It will be appreciated that X (the replaced/modified differentiated amino acids) could be any naturally occurring amino acid. Furthermore it could be a synthetic amino acid analogue.

Although Freund's complete and incomplete adjuvant generally elicits the strongest immune response, other adjuvants may be acceptable such as Quil A, Gerbu adjuvants, Titermax adjuvants or alum.

For seven different peptide sequences we have shown in secret trials improved antibody production in sheep, elicited by randomised peptides compared to control peptides lacking the random amino acids. We have subsequently raised antisera successfully with many other randomised peptides.

In the peptides prepared to date the “soup” or “cocktail” used for the random substitution has been made including all amino acids at equimolar concentrations. The different amino acids substitute with differentiating efficiencies and this is dependent upon the preceding amino acids and the conformation adopted by the peptide. It would, therefore, be difficult to adjust the concentrations in the “soup” to produce equimolar substitution for every different peptide. We start with equimolar concentrations of the different amino acids that will substitute X, and it does not matter that the resultant soup of peptides has differently modified peptides at different concentrations—a good result when administered is still achieved.

There is, however, a theoretical basis for arguing that using a smaller number of amino acids, say <15 or <20 amino acids, in the “soup” or “cocktail” when creating differential peptides could give improved results due to a threshold concentration of any particular sequence being required for T-cell recruitment. We have used peptides where there were only four (L, F, D and Q) or eight (L, F, D, Q, P, E, A, and Y) amino acids in the “soup”. Both strategies worked but the eight amino acids appeared slightly better and we prefer to use eight amino acids for the substituted peptides.

A typical immunisation schedule would use a 100-1,000 μg (e.g. 500 μg) prime followed by a 50-500 μg (e.g. 200 μg) boost after six weeks (for an adult patient). If necessary, subsequent boost injections could be given at monthly intervals but this should, ideally, be determined by clinical indices and will differ from patient to patient.

It may in certain circumstances not be necessary to administer an adjuvant with the target-differentiated peptides. We may provide a medicament with or without an adjuvant. The user may add one later, or may not.

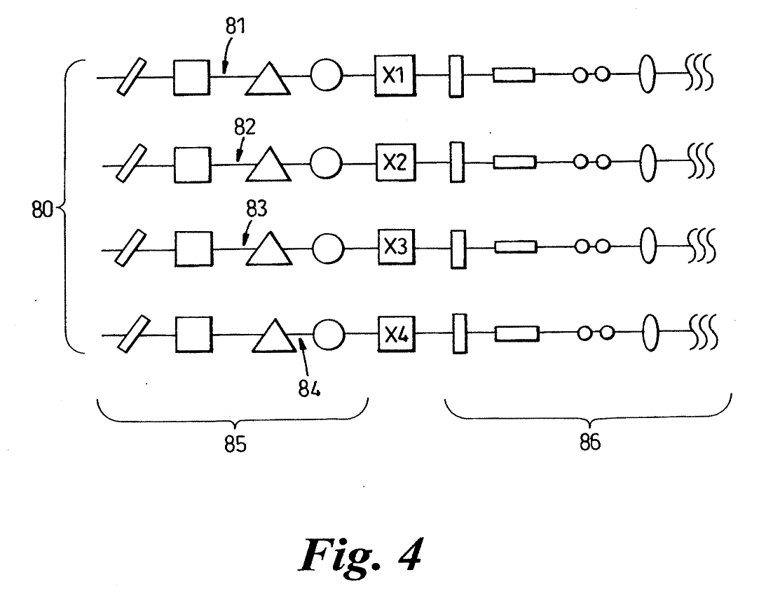

FIG. 4 shows a composition 80 having four variants, 81, 82, 83, 84, each with a different amino acid substituted at X:X1, X2, X3, and X4, respectively (each of which is different from the amino acid in the target molecule at the site X). The composition has variants 81-84 in generally equi-molar concentrations and was made by taking the left-hand part of the peptide chain, referenced 85, (or the right-hand end, referenced 86) and adding a mixture of amino acids X1, X2, X3, X4 in equimolar concentrations to the reaction mixture to add the next amino acid to the chain. The other half/part 86 (or 85) is then added to the reaction mixture and joined to the amino acid X1, X2, X3, or X4, producing four variants that differ by X1, X2, X3 or X4.

Alternatively, instead of grafting complete sections of peptide chain to the variant amino acid the variants 81-84 could be built up amino acid by amino acid (or one half could be pre-formed and the other stepwise fabricated).

### EXAMPLES

### Experimental Verification

The following peptides had been used, with some success, as eight-branch multi-antigenic-peptides for raising antibodies in sheep. Modifying the peptides by inclusion of one or more random substitutions improved the antibody response produced.

All immunisations were performed in a standard manner with 500 μg of peptide in Freund's complete adjuvant for prime and 200 μg in Freund's incomplete adjuvant for boosts at six weeks. Test bleeds were assessed by radial immunodiffusion (Mancini) so the quality (strength) of the precipitation is correlated by the avidity of the antibody (scored as weak, moderate or strong) while the diameter of the precipitation is inversely proportional to the titre of the antibody.

CD13

The following (unmodified) peptide was used in three different sheep but produced no antibody response or, at best, weak (mean diameter 11.5 mm) responses even after three reboosts in two of the animals.

(SEQ ID NO: 14)

F K Q W M E N P N N N P I H P N L R S T V

(SEQ ID NO: 15)

F K Q W X E N P N N N P I H P N L R S T V

Using a pool of twenty amino acids at the positions marked X, a strong response (9 mm against the unmodified peptide) was produced after the first boost in a fourth animal.

CD61

The following (unmodified) peptide was used in two different sheep but produced no antibody response or, at best, weak (10, 6.2 mm) responses in one of the animals.

| | |
|---|---|
| G P N I C T T R G V S S C Q Q | (SEQ ID NO: 16) |
| G P X I C T T R G V S S X Q Q | (SEQ ID NO: 17) |

Using a pool of twenty amino acids at the positions marked X, a strong response (9 mm) was produced after the first boost in a third animal.

HCG#1

The following (unmodified) peptide was used in two different sheep but produced no antibody response or, at best, weak (mean 13.5 mm) responses in one of the animals.

(SEQ ID NO: 18)

K A P P P S L P S P S R L P G P S D T P I L P Q

(SEQ ID NO: 19)

K X P P P S L P S P S R L P G P S D T P I L P Q

Using a pool of twenty or four amino acids at the position marked X, produced strong (12 and 10.3 mm respectively) responses in two further sheep.

HGC#2

The following (unmodified) peptide was used in two different sheep but produced no antibody response.

(SEQ ID NO: 20)

D P R F Q D S S S S K A P P P S L P S P S R L P

(SEQ ID NO: 21)

D P X F Q D S S S S X A P X P S L P S P S X L P
1 2 3 4

Using a pool of twenty amino acids at the selected ones of the numbered positions indicated produced the following improved responses:

At Position 1=strong (10.2 mm).

At Position 4—weak (not measurable).

At Position 1+4—weak (not measurable).

At Position 2—weak/moderate (15.6 mm).

At Position 3—strong (12.5 mm).

Using a pool of four or eight amino acids at position 1 also produced strong responses (12.3 and 9.7 mm respectively).

Myoglobin

The following (unmodified peptide did produce a reasonable antibody response in a sheep (8.8 mm) but extension of the peptide with inclusion of a randomised position (twenty amino acids) improved the response (8.4 mm) and the antibodies produced were also found to bind to the native protein in immunohistology.

(SEQ ID NO: 22)

QSKHPGDFGADAQGAMNKALQVXQSKHPDFGADAQGAMNKAL

It will be appreciated that the present invention can be considered to be the creation of an improved immune response in an animal by administering, for example injecting, a cocktail of peptides that are similar to, but different from, a target peptide against which it is desired to create antibodies. The similar peptides are preferably a cocktail of peptides having the same peptide sequence as the target molecule, but with one or more amino acids substituted for a chance-selected, random, amino acid from a group of allowable amino acids.

An adjuvant and/or the target molecule itself may be injected with the cocktail of similar peptides. There may be substantially no other substances in the vaccine preparation to be injected which elicits a substantial immune response (or substantially any immune response) beyond the cocktail of similar proteins, and optionally the adjuvant and/or target molecules. The preparation/vaccine may comprise an immunologically acceptable carrier, such as water. The similar peptides may be present in the vaccine/preparation that is administered at a level of 100-1,000 μg in total peptides, excluding the adjuvant. The preparation/vaccine is preferably administered as a single injection. Alternatively we may use multiple site injections (e.g. small doses of vaccine injected at different sites on the human or animal body).

The peptides in the preparation/medicament are preferably provided as multiple branches attached to a common molecule, or core. There may be 4, 8, or 16 branches (or another number).

1. A composition for eliciting an immune response against a target protein, the composition comprising a plurality of synthesised peptide ingredients comprising a first peptide substantially homologous to a first portion of the target protein except for an amino acid in at least one amino acid position in the first peptide being randomly-selected from a pool consisting of naturally occurring amino acids and/or amino acid analogs.

2. The composition of claim 1, wherein the at least one amino acid position in the first peptide is at least one and up to four amino acid positions.

3. The composition of claim 1, further comprising one or more of an ingredient selected from the group consisting of an adjuvant, an excipient, a solvent and a carrier.

4. The composition of claim 1, wherein the pool comprises between two and about twenty amino acids.

5. The composition of claim 1, wherein the pool comprises between two and about twenty amino acid analogs.

6. The composition of claim 1, wherein the synthesised peptide is between about ten and about 50 amino acids in length.

7. The composition of claim 1, wherein the synthetic peptide ingredients comprise a multiple antigen peptide system.

8. The composition of claim 7, wherein the multiple antigen peptide system is divalent, tetravalent, octavalent or hexadecavalent.

9. The composition of claim 7, wherein the multiple antigen peptide system comprises a lysine core.

10. The composition of claim 1, wherein the plurality of synthesised peptide ingredients further comprises a second peptide, the second peptide being substantially homologous to a second portion of the target protein except for an amino acid in at least one amino acid position in the second peptide being randomly selected from the pool.

11. The composition of claim 10, wherein the at least one amino acid position in the second peptide is at least one and up to four amino acid positions.

12. The composition of claim 10, wherein the plurality of synthesised peptide ingredients further comprises a third or more peptide, the third or more peptide being substantially homologous to a third or more portion of the target protein except for an amino acid in at least one amino acid position in the third or more peptide being randomly selected from the pool.

13. The composition of claim 12, wherein the at least one amino acid position in the third or more peptides is at least one and up to four amino acid positions.

14. A composition for eliciting an immune response against a target protein, the composition comprising a plurality of synthesised peptide ingredients, each ingredient being adapted for eliciting an immune response against the target protein, by the steps of:
i) synthesising a first peptide substantially homologous to a portion of the target protein, wherein in at least one amino acid position an amino acid is taken at random from a pool consisting of naturally occurring amino acids and amino acid analogs;
ii) obtaining a plurality of the first peptide, wherein in each of the plurality of the first peptide the amino acid in the at least one amino acid position is taken at random from the pool; and
iii) providing the plurality of the first peptide in a suitable composition.

15. The composition of claim 14, wherein the peptide is grown on a multiple antigen peptide system.

16. A kit comprising the composition of claim 1 and instructions for use.


SEQUENCE LISTING (22 sequences)

SEQ ID NO: 1 - protein, 34 aa, Homo sapiens
  SITE (2)..(2): X= A, R, N, D, C, Q, E, G, H, I, L, K, M, F, P, S, T, W, Y, V
SXSEIQLMHNLGKHLNSMERVEWLRKKLQDVHNF

SEQ ID NO: 2 - protein, 20 aa, Homo sapiens
  SITE (4)..(4): X= A, R, N, D, C, Q, E, G, H, I, L, K, M, F, P, S, T, W, Y, V
NFVXLGAPLAPRDAGSQRPR

SEQ ID NO: 3 - protein, 34 aa, Homo sapiens
  SITE (4)..(4): X= A, R, N, D, C, Q, E, G, H, I, L, K, M, F, P, S, T, W, Y, V
PRKXEDNVLVESHEKSLGEADKADVNVLTKAKSQ

SEQ ID NO: 4 - protein, 24 aa, Homo sapiens
  SITE (4)..(4): X= A, R, N, D, C, Q, E, G, H, I, L, K, M, F, P, S, T, W, Y, V
SLQXVFGIELMEVDPIGHLYIFAT

SEQ ID NO: 5 - protein, 25 aa, Homo sapiens
  SITE (4)..(4): X= A, R, N, D, C, Q, E, G, H, I, L, K, M, F, P, S, T, W, Y, V
YRQXPGSDPACYEFLWGPRALVETS

SEQ ID NO: 6 - protein, 40 aa, Homo sapiens
  SITE (27)..(27): X= A, R, N, D, C, Q, E, G, H, I, L, K, M, F, P, S, T, W, Y, V
MPREDAHFIYGYPKKGHGHSYTTAEEXAGIGILTVILGVL

SEQ ID NO: 7 - protein, 30 aa, Homo sapiens
  SITE (5)..(5): X= A, R, N, D, C, Q, E, G, H, I, L, K, M, F, P, S, T, W, Y, V
QEKNXEPVVPNAPPAYEKLSAEQSPPPYSP

SEQ ID NO: 8 - protein, 27 aa, Homo sapiens
  SITE (10)..(10): X= A, R, N, D, C, Q, E, G, H, I, L, K, M, F, P, S, T, W, Y, V
MLLAVLYCLXWSFQTSAGHFPRACVSS

SEQ ID NO: 9 - protein, 25 aa, Homo sapiens
  SITE (4)..(4): X= A, R, N, D, C, Q, E, G, H, I, L, K, M, F, P, S, T, W, Y, V
KQLXEEKQPLLMEKEDYHSLYQSHL

SEQ ID NO: 10 - protein, 30 aa, Homo sapiens
  SITE (13)..(13): X= A, R, N, D, C, Q, E, G, H, I, L, K, M, F, P, S, T, W, Y, V
DGGPCPSGSWSQXRSFVYVWKTWGQYWQVL

SEQ ID NO: 11 - protein, 30 aa, Homo sapiens
  SITE (11)..(11): X= A, R, N, D, C, Q, E, G, H, I, L, K, M, F, P, S, T, W, Y, V
TIMDQVPFSVXVSQLRALDGGNKHFLRNQP

SEQ ID NO: 12 - protein, 28 aa, Homo sapiens
  SITE (6)..(6): X= A, R, N, D, C, Q, E, G, H, I, L, K, M, F, P, S, T, W, Y, V
NFATGXNECDVCTDQLFGAARPDDPTLI

SEQ ID NO: 13 - protein, 25 aa, Homo sapiens
  SITE (5)..(5): X= A, R, N, D, C, Q, E, G, H, I, L, K, M, F, P, S, T, W, Y, V
HYYSXRDTLLGPGRPYRAIDFSHQG

SEQ ID NO: 14 - protein, 21 aa, Homo sapiens
FKQWMENPNNNPIHPNLRSTV

SEQ ID NO: 15 - protein, 21 aa, Homo sapiens
  SITE (5)..(16): X= A, R, N, D, C, Q, E, G, H, I, L, K, M, F, P, S, T, W, Y, V
FKQWXENPNNNPIHPXLRSTV

SEQ ID NO: 16 - protein, 15 aa, Homo sapiens
GPNICTTRGVSSCQQ

SEQ ID NO: 17 - protein, 15 aa, Homo sapiens
  SITE (3)..(13): X= A, R, N, D, C, Q, E, G, H, I, L, K, M, F, P, S, T, W, Y, V
GPXICTTRGVSSXQQ

SEQ ID NO: 18 - protein, 24 aa, Homo sapiens
KAPPPSLPSPSRLPGPSDTPILPQ

SEQ ID NO: 19 - protein, 24 aa, Homo sapiens
  SITE (2)..(2): X= A, R, N, D, C, Q, E, G, H, I, L, K, M, F, P, S, T, W, Y, V
KXPPPSLPSPSRLPGPSDTPILPQ

SEQ ID NO: 20 - protein, 24 aa, Homo sapiens
DPRFQDSSSSKAPPPSLPSPSRLP

SEQ ID NO: 21 - protein, 24 aa, Homo sapiens
  SITE (3)..(22): X= A, R, N, D, C, Q, E, G, H, I, L, K, M, F, P, S, T, W, Y, V
DPXFQDSSSSXAPXPSLPSPSXLP

SEQ ID NO: 22 - protein, 42 aa, Homo sapiens
  SITE (23)..(23): X= A, R, N, D, C, Q, E, G, H, I, L, K, M, F, P, S, T, W, Y, V
QSKHPGDFGADAQGAMNKALQVXQSKHPDFGADAQGAMNKAL